(19) United States
(12) Patent Application Publication
(10) Pub. No.: US 2017/0007456 A1
(43) Pub. Date: Jan. 12, 2017

(54) SYSTEM FOR INCREASING THE NUMBER OF FOCAL POINTS IN ARTIFICIAL EYE LENSES

(71) Applicant: ISTANBUL TEKNIK ÜNIVERSITESI, Istanbul (TR)

(72) Inventors: Selcuk AKTURK, Istanbul (TR); Rifat RASIER, Istanbul (TR)

(21) Appl. No.: 15/115,134

(22) PCT Filed: May 21, 2015
(86) PCT No.: PCT/TR2015/000206

§ 371 Date: Jul. 28, 2016

(30) Foreign Application Priority Data
Mar. 12, 2015 (TR) 2015/02944

(51) Int. Cl. A61F 9/008

(52) U.S. Cl. CPC A61F 9/00834; A61F 2240/001; A61F 2/1618

(57) ABSTRACT

A system for increasing the number of focal points in artificial eye lenses has a light source that produces a pulsed light via a beam controller that shapes the light beam according to the type of the application and properties of the zone plates that will be produced. A pulse controller conditions the light pulses according to the type of predetermined application and the zone plates that will be produced. A beam director directs the light to predetermined parts of the surface of the artificial eye lens where the zone plates will be produced. A focusing unit focuses the light beam by reducing its diameter. A scanning unit produces zone plates on the artificial eye lens by scanning the light beam on the predetermined parts of the surface of the artificial eye lens where the zone plates will be produced.

6

### FIELD OF THE INVENTION

The present invention relates to increasing the number of focal points of eye lenses by using a light source in order to correct vision defects.

### BACKGROUND OF THE INVENTION

Cataract, which is one of the most common eye diseases, is formed when the natural lens of the eye gets dense and loses its transparency and acquires an opaque, cloudy appearance. Since there is no cure for cataract with drugs or eyeglasses, patients can be treated only by a surgical operation. The surgical operation is based on the principle of removing the natural lens of the eye, which has lost its transparency, and replacing it with a new artificial intraocular lens.

The most important deficiency of intraocular lenses used in cataract surgeries is the fact that the patient cannot change the focal point of the intraocular lens. A healthy natural eye lens has a transparent and elastic form. By means of this elasticity, the ciliary muscles attached to the natural lens which can contract and relax can change the focal point of the lens by changing the shape of the lens. Thus, the individual can clearly see objects that are at a close, medium and far distance (this capability is called accommodation). Since intraocular lenses are not attached to the ciliary muscles, the patient cannot change the focal point of the lens and thus lacks the accommodation capability. As a result, since a patient who has a monofocal intraocular lens implanted by a surgical operation can focus on only one of the far, medium and close distances, s/he can acquire visibility only at the focused distance. The patient cannot have visibility at the other unfocused distances and will have to use glasses for these distances.

However, especially in the recent years, multifocal lenses are started to be used. Since it is possible to focus on a plurality of distances with the multifocal lenses, the patient can see far, medium and close distances without using glasses. The structure of an intraocular multifocal lens may change according to the production method, but it is generally formed by forming special cross-sections at different regions of the lens surface. The multifocal lenses are produced according to two different principles named as refractive and diffractive lenses.

In the refractive method, different focal lengths are obtained by changing the surface curvature in different regions of the lens. A method named Fresnel lens is used for decreasing thickness of the lens. In this method, the surface of the lens is divided into circular regions and curvature of each region is designed to be equal to that of the spherical lens that is taken as basis. Thus the lens is “folded” in steps.

In the diffractive method, depending on the principle of a system named Fresnel zone plate, rings having different diameters and depths are formed on the artificial lens. In the case that a zone plate is produced on the surface of a spherical lens, the light coming onto this lens exhibits two different focusing behaviors. The primary focus is formed with the refractive effect resulting from the curved surfaces of the lens and this is called zeroth-order. The secondary focus is formed at the point determined by the ring radii as a result of the behavior of the zones at the Fresnel zone plate and this point is named as first-order. This way the lens exhibits multifocal behavior.

There are many patients who have undergone a cataract surgery in which a monofocal lens is placed in the eye. This, as stated before, requires the patient to use an auxiliary apparatus such as glasses or contact lenses. One solution for eliminating this requirement is possible by replacing the monofocal lens which is previously implanted in the eye with a multifocal lens. However, removing a monofocal lens in the eye is an extremely difficult and risky surgical operation.

In addition to this, multifocal lenses have production methods which are difficult and thus costly requiring high precision due to complicated surface profiles of the multifocal lenses. Due to the fact that multifocal lenses are 10 times more costly than monofocal lenses, patients may prefer monofocal lenses over multifocal lenses.

In another case, patients, who think that they will suffer side effects of multifocal lenses such as shading, ghosting and halo problems, may prefer monofocal lenses.

If patients who have preferred monofocal lenses before due to various reasons want to replace them with multifocal lenses, they have to go through a difficult and risky surgical operation for removal of the monofocal lens from within the eye.

### SUMMARY OF THE INVENTION

The objective of one embodiment of the present invention is to increase the number of focal points of an artificial eye lens by the help of a light source.

The objective of one embodiment of the present invention is to increase the number of focal points of an intraocular lens by the help of a light source.

The objective of one embodiment of the present invention is to increase the number of focal points of an intraocular lens by the help of a light source while the lens is in the eye.

### DETAILED DESCRIPTION OF THE INVENTION

In order for an example embodiment of the present system for increasing the number of focal points in artificial eye lenses to be understood better, the said embodiment is illustrated in the figures and the details of the embodiments of the said system should be evaluated taking into consideration the entire description. The mentioned figures are as follows:

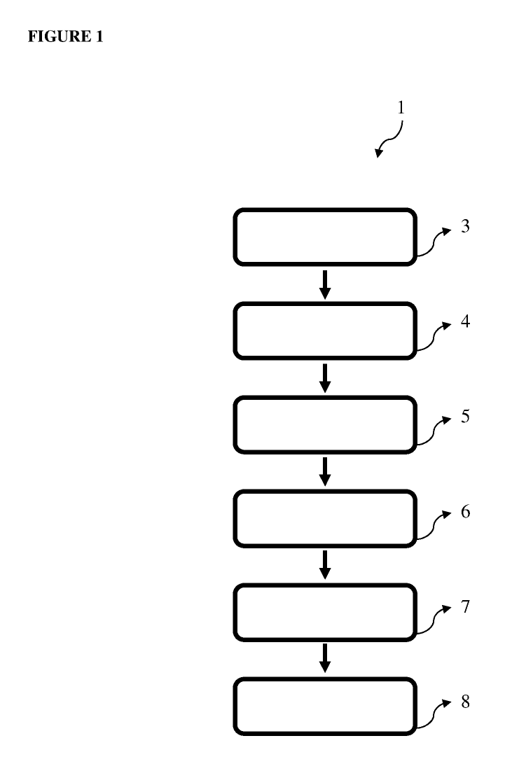

FIG. 1 is a schematic view of one embodiment of the system of increasing the number of focal points in artificial eye lenses.

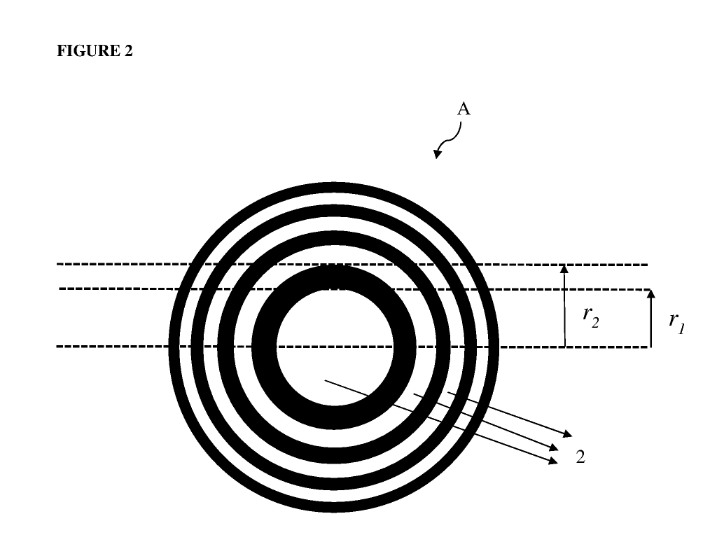

FIG. 2 is a schematic view of an artificial eye lens on which zone plates are produced.

The components in the figures are given reference numbers as follows:
1. System of increasing the number of focal points in eye lenses
2. Zone plate
3. Light source
4. Beam controller
5. Pulse controller
6. Beam director
7. Focusing unit
8. Scanning unit
A. Artificial eye lens
r₁, r₂ Example radii of zone plates (2) calculated approximately relative to a predetermined center

A system of increasing the number of focal points in artificial eye lenses (1), which enables to increase the number of focal points of an artificial eye lens (A) by producing zone plates (2) on the artificial eye lens (A), basically comprises
at least one light source (3) which produces a pulsed light,
at least one beam controller (4) which can shape the light beam coming from the light source (3) according to the type of the application and properties of the zone plates (2) that will be produced,
at least one pulse controller (5) which conditions the light pulses produced by the light source according to the type of predetermined application and the zone plates (2) that will be produced,
at least one beam director (6) which directs the light to the predetermined parts of the surface of the artificial eye lens (A) where the zone plates (2) will be produced,
at least one focusing unit (7) which focuses the light beam by reducing its diameter,
at least one scanning unit (8) which produces zone plates (2) on the artificial eye lens (A) by scanning the light beam on the predetermined parts of the surface of the artificial eye lens (A) where the zone plates (2) will be produced,
a light source (3) which produces light in the wavelength range in which the eye's cornea is transparent,
at least one interface, which is fixed on the eye where the artificial eye lens (A), whose focal points are desired to be increased, is located, and which prevents movement of the eye,
the artificial eye lens (A) being a contact lens,
the artificial eye lens (A) being an intraocular lens,
the artificial eye lens (A) being an intraocular lens which is previously implanted in an eye, and
a scanning unit (8) which produces zone plates (2) on the said intraocular lens while the intraocular lens is still in the eye.

The system of increasing the number of focal points in artificial eye lenses (1) of the present invention relates to increasing the number of focal points of an artificial eye lens (A) by the help of a light source (3). The system of increasing the number of focal points in artificial eye lenses (1) is based on the principle of producing zone plates (2) on the artificial eye lenses (A) by the help of a pulsed light coming from a light source (3) thereby increasing the number of focal points of the artificial eye lens (A). An artificial eye lens (A), whose focal points are increased by producing zone plates (2), can focus the incoming light to a plurality of points at the same time. Any lens which is to be used for improving vision disorder of a patient suffering a vision disorder is named as an artificial eye lens (A). Generally intraocular lenses and contact lenses can be given as examples to the artificial eye lenses (A), however artificial eye lenses (A) are not limited with these examples.

In a preferred embodiment of the invention, a laser source is used as the light source (3). The desired zone plates (2) are produced at the predetermined parts of the artificial eye lenses (A) upon processing the surfaces of the artificial eye lenses (A) precisely by the light source (3) which is a laser. In a preferred embodiment of the invention, the light source (3) is a pulsed laser source. Nanosecond, picosecond or femtosecond pulsed lasers can be used as pulsed laser source but it is not limited with those. In a preferred embodiment of the invention, the femtosecond pulsed laser having the highest precision is used as the light source (3).

The zone plates are comprised of regions which are in the form of concentric rings. The surface, on which the zone plate will be produced, is divided into radii approximately calculated with the formula of rₙ=\sqrt{nfλ} relative to a predetermined center. In this formula, n is an integer which determines the radius (starts from 1 and increases), rₙ is the n^{th} radius, f is the effective focus distance that will be formed, and λ is the wavelength of the light that will be focused. Each one of the areas which remain between two neighboring radii and which is circular in the center and ring-shaped around the center is called a zone plate (2) (FIG. 2). After the zone plates are determined this way, optical properties thereof are changed consecutively. The zones are adjusted such that the center zone is fully transparent, the first neighbor is fully opaque, and the next one is fully transparent (and will continue this way). This form is called binary. As an alternative, light transparency between the neighboring zones is changed gradually. As another alternative, thicknesses of the neighboring zones are adjusted such that the difference therebetween is approximately half of a wavelength. In a preferred embodiment of the invention, thicknesses and light transparencies of the zones determined on the artificial eye lens (A) are changed by the light source (3).

The light which is output from the light source (3) and which is preferably a femtosecond pulsed laser is in the form of a beam, and passes through the beam controller (4) and the beam director (6).

The beam controller (4) can narrow, expand or shape the light beam according to the type of the system of increasing the number of focal points in artificial eye lenses (1) and the properties (e.g. radius) of the zone plates (2) that will be produced. Mirrors, lenses or computer controlled modulators can be used as beam controller (4).

A pulse controller (5), which can be located before or after the light beam controller (4), conditions the light pulses produced by the light source (3) according to the type of the predetermined application type and the zone plates (2) that will be produced. In the pulse controller (5), processes such as pulse selection, pulse stretching, pulse compression are applied to the light beam according to the type of the predetermined application and the zone plates (2) that will be produced. Pulse selection is performed by passing one or more predetermined light pulses and preventing others among the light pulses in the pulse train produced consecutively from the light source (3). Pulse stretching and compression is performed by increasing or decreasing the total period of time in which a pulse is active (pulse width). In the preferred embodiment of the invention, the light pulse is a femtosecond laser pulse.

The beam director (6) directs the light, which is beam controlled (in the beam controller (4)) and pulse controlled (in the pulse controller (5)), to the predetermined parts of the surface of the artificial eye lens (A) where the zone plates (2) will be produced. The beam director (6) may be comprised of articulated mirror arms, mirror arms placed in tubular light paths having various numbers of articulations and mirrors placed in articulations, or flexible fibers in which light is guided and transmitted.

The light is delivered from the beam director (6) to a focusing unit (7). The focusing unit (7) focuses the light beam by reducing its diameter. Structures comprised of components with lenses and mirrors can be used as the focusing unit (7). The light beam, which is focused by reducing its diameter in the focusing unit (7), is transmitted to a scanning unit (8). The scanning unit (8) scans (directs) the focused light beam on the predetermined parts of the surface of the artificial eye lens (A) where the zone plates (2) will be produced. Thus, zone plates (2) are produced on the predetermined parts of the artificial eye lens (A).

In one embodiment of the invention, the artificial eye lens (A) is a contact lens and the number of focal points of the contact lens is increased by producing zone plates (2) on the contact lens. Production of multifocal contact lenses becomes easier and more inexpensive by producing zone plates (2) on the contact lens by using the system of increasing the number of focal points in the artificial eye lenses (A). In addition, a monofocal contact lens which is produced previously can be converted to a multifocal contact lens.

In one embodiment of the invention, the artificial eye lens (A) is an intraocular lens and the number of focal points of the intraocular lens is increased by producing zone plates (2) on the intraocular lens. Production of multifocal intraocular lenses becomes easier and more inexpensive by producing zone plates (2) on the intraocular lens by using the system of increasing the number of focal points in the artificial eye lenses (A). In addition, a monofocal intraocular lens which is produced previously can be converted to a multifocal intraocular lens.

In one embodiment of the invention, the artificial eye lens (A) is an intraocular lens which is previously implanted in the eye of the patient and the number of focal points of the said intraocular lens is increased by producing zone plates (2) on the said intraocular lens while the intraocular lens is in the eye of the patient. Production of multifocal intraocular lenses becomes easier and more inexpensive by producing zone plates (2) on the intraocular lens by using the system of increasing the number of focal points in the artificial eye lenses (A). In addition, a monofocal intraocular lens which is produced previously can be converted to a multifocal intraocular lens while it is in the patient's eye. Thus, the patient can focus on a plurality of distances and can have visibility in these distances without having to replace the intraocular lens that is located in the patient's eye. This process saves the patient from a surgical operation for replacing the intraocular lens and from using auxiliary apparatuses such as glasses and contact lenses required for providing visibility at distances which cannot be focused by the monofocal lens.

In the embodiment of the invention in which zone plates (2) are produced in the intraocular lens located in the patient's eye, the wavelength of the light produced by the light source (3) is in the wavelength range in which the eye's cornea is transparent. Therefore, in the embodiments of the invention in which zone plates (2) are produced in the intraocular lens located in the patient's eye, the wavelength of the light source (3) is in visible and infrared regions. In the other embodiments of the invention, when producing zone plates (2) on the intraocular lens and contact lens, there is no limitation for the wavelength of the light source (3).

In the embodiments of the invention in which zone plates (2) are produced in the intraocular lens located in the patient's eye, an interface can be used to fix the eye (and thus the intraocular lens). The interface is preferably fixed on the eye via vacuum and prevents movement of the eye during the procedures. The light passing through the interface produces the zone plates (2) on the intraocular lens. Thus, when the zone plates (2) are being produced on the predetermined parts (positions) on the artificial eye lens, dislocations and errors that may occur in the positions of the zone plates (2) due to the eye's movement can be prevented by the interface.

1. A system of increasing the number of focal points in artificial eye lenses (1), which enables to increase the number of focal points of an artificial eye lens (A) by producing zone plates (2) on the artificial eye lens (A), basically comprising
at least one light source (3) which produces a pulsed light, and
at least one beam controller (4) which can shape the light beam coming from the light source (3) according to the type of the application and properties of the zone plates (2) that will be produced,
at least one pulse controller (5) which conditions the light pulses produced by the light source according to the type of predetermined application and the zone plates (2) that will be produced,
at least one beam director (6) which directs the light to the predetermined parts of the surface of the artificial eye lens (A) where the zone plates (2) will be produced,
at least one focusing unit (7) which focuses the light beam by reducing its diameter,
at least one scanning unit (8) which produces zone plates (2) on the artificial eye lens (A) by scanning the light beam on the predetermined parts of the surface of the artificial eye lens (A) where the zone plates (2) will be produced.

2. A system of increasing the number of focal points in artificial eye lenses (1) according to claim 1, comprising a light source (3) which produces light in the wavelength range in which the eye's cornea is transparent.

3. A system of increasing the number of focal points in artificial eye lenses (1) according to claim 1, comprising at least one interface, which is fixed on the eye where the artificial eye lens (A), whose focal points are desired to be increased, is located, and which prevents movement of the eye.

4. A system of increasing the number of focal points in artificial eye lenses (1) according to claim 1, wherein the artificial eye lens (A) is a contact lens.

5. A system of increasing the number of focal points in artificial eye lenses (1) according to claim 1, wherein the artificial eye lens (A) is an intraocular lens.

6. A system of increasing the number of focal points in artificial eye lenses (1) according to claim 2, wherein the artificial eye lens (A) is an intraocular lens which is previously implanted in an eye, and comprising a scanning unit (8) which produces zone plates (2) on said intraocular lens while the intraocular lens is still in the eye.

7. A system of increasing the number of focal points in artificial eye lenses (1) according to claim 1 comprising a scanning unit (8) which, by producing zone plates (2) on a monofocal artificial eye lens (A), produces a multifocal artificial eye lens from a monofocal artificial eye lens (A).

8. A system of increasing the number of focal points in artificial eye lenses (1) according to claim 2 comprising a scanning unit (8) which, by producing zone plates (2) on a monofocal artificial eye lens (A), produces a multifocal artificial eye lens from a monofocal artificial eye lens (A).

9. A system of increasing the number of focal points in artificial eye lenses (1) according to claim 3 comprising a scanning unit (8) which, by producing zone plates (2) on a monofocal artificial eye lens (A), produces a multifocal artificial eye lens from a monofocal artificial eye lens (A).

10. A system of increasing the number of focal points in artificial eye lenses (1) according to claim 4 comprising a scanning unit (8) which, by producing zone plates (2) on a monofocal artificial eye lens (A), produces a multifocal artificial eye lens from a monofocal artificial eye lens (A).

11. A system of increasing the number of focal points in artificial eye lenses (1) according to claim 5 comprising a scanning unit (8) which, by producing zone plates (2) on a monofocal artificial eye lens (A), produces a multifocal artificial eye lens from a monofocal artificial eye lens (A).

12. A system of increasing the number of focal points in artificial eye lenses (1) according to claim 6 comprising a scanning unit (8) which, by producing zone plates (2) on a monofocal artificial eye lens (A), produces a multifocal artificial eye lens from a monofocal artificial eye lens (A).

13. A method of increasing focal point of an intraocular lens already implanted in the eye of a human, comprising the steps of:
providing a laser adjacent an intraocular lens, and
forming a plurality of zone plates on the lens by applying laser light from the laser onto an outward surface of the intraocular.

14. A method according to claim 13, including the step of applying laser light having a wavelength in the visible and infrared regions

15. A method according to claim 13, including the step of forming the zone plates as concentric rings, each ring having a radius calculated according to the following formula
r₂=\sqrt{nfλ}
where n is an integer determining the radius in order, rₙ is the n^{th} radius, f is the effective focus distance to be formed, and λ is the wavelength of the laser light to be focused.
